(19) United States
(12) Patent Application Publication
(10) Pub. No.: US 2021/0308073 A1
(43) Pub. Date: Oct. 7, 2021

(54) COMPOSITIONS COMPRISING PROPOFOL, KETAMINE, AND ANALGESIC, AND METHODS OF USE

(71) Applicant: THE REGENTS OF THE UNIVERSITY OF CALIFORNIA, Oakland, CA (US)

(72) Inventor: Zhuang Ting Fang, Arcadia, CA (US)

(21) Appl. No.: 17/265,036

(22) PCT Filed: Aug. 1, 2019
(86) PCT No.: PCT/US19/44612

§ 371 Date: Feb. 1, 2021

Related U.S. Application Data
(60) Provisional application No. 62/713,087, filed on Aug. 1, 2018.

(51) Int. Cl. A61K 31/05; A61K 31/135; A61K 31/454; A61K 31/4468; A61K 31/485; A61K 31/4535; A61K 31/4174; A61K 9/08; A61K 9/00; A61P 25/20; A61P 25/04

(52) U.S. Cl. CPC A61K 31/05; A61K 31/135; A61K 31/454; A61K 31/4468; A61K 31/485; A61P 25/04; A61K 31/4174; A61K 9/08; A61K 9/0019; A61P 25/20; A61K 31/4535

(57) ABSTRACT

The present invention provides pharmaceutical compositions comprising propofol, ketamine, and analgesics and methods for treating a subject with the pharmaceutical composition. In one aspect, the present invention relates to methods of treating a subject undergoing surgery under MAC (monitored anesthesia care) with the pharmaceutical composition.

### CROSS-REFERENCE TO RELATED APPLICATIONS

The present application claims priority to U.S. Provisional Application Ser. No. 62/713,087, filed on Aug. 1, 2018, which is incorporated by reference herein in its entirety.

### BACKGROUND OF THE INVENTION

In addition to general anesthesia and regional anesthesia, Monitored Anesthesia Care (MAC) is one of the three techniques of anesthesia practice. Because of the side effects of the sedatives commonly used in MAC, including respiratory and cardiovascular depression, delivering those medications intravenously to provide an adequate level of sedation, profound analgesia, minimum risk of complications in a timely predictable fashion is a major challenge. When complications occur, the risk of patient injury for MAC is as high as that of general anesthesia, leading to significant morbidity and mortality. There are various techniques of MAC intravenous sedation. The majority of anesthesia providers use combinations of sedatives, in different syringes, from different categories of medications, including propofol, midazolam, narcotics (fentanyl, alfentanil, remifentanil, sufentanil), ketamine, sodium thiopantol, and dexmedetomidine. It is quite cumbersome to give multiple drugs in different syringes at the same time. In addition, the potential side effects can be additive too, such as respiratory depression from the inhibition of mu-receptor by narcotics and the stimulation of the GABA receptor by propofol and midazolam. If one delivers all these drugs too fast, the risk of complication increases. On the other hand, if one titrates the drugs slowly, it might be safer, but it increases the time for sedation, which would not meet the financial economic demand of the anesthesia practice, especially in the setting of fast-track ambulatory surgery and anesthesia. Even with the approach of slow titration, the analgesic portion of the sedation is still not reliably predictable leading to pain and patient movement which is one of major causes of needle injury during nerve blocks, such as retrobulbar block. The outcome is usually poor, including permanent vision loss.

Thus, there remains a need in the art for a novel, efficacious, and safe technique for intravenous sedation in anesthesia practice. The present invention addresses this unmet need.

### SUMMARY OF THE INVENTION

In one aspect, the present invention relates to a pharmaceutical composition comprising a solution of propofol, ketamine, and an analgesic wherein the concentration of propofol is 1 mg/mL-10 mg/mL, the concentration of ketamine is 0.1 mg/mL-10 mg/mL, and the concentration of analgesic is 1 μg/mL-1 mg/mL. In one embodiment, the analgesic is selected from the group consisting of alfentanil, fentanyl, remifentanil, hydromorphone, sufentanil, morphine, and dexmedetomidine. In one embodiment, the analgesic is fentanyl. In one embodiment, the composition comprises 5 mg/mL-7 mg/mL propofol, 1 mg/mL-3 mg/mL ketamine, and 5 μg/mL-0.02 mg/mL analgesic. In one embodiment, the solution comprises a sterile medium selected from the group consisting of water, deionized water, distilled water, sterilized water, saline solution, dextrose in water solution, and Ringer's lactate solution.

The present invention also relates in part to a method of providing sedation to a subject in need thereof, the method comprising the step of administering to the subject a pharmaceutical composition comprising a solution of propofol, ketamine, and an analgesic wherein the concentration of propofol is 1 mg/mL-10 mg/mL, the concentration of ketamine is 0.1 mg/mL-10 mg/mL, and the concentration of analgesic is 1 μg/mL-1 mg/mL; and wherein the composition is administered in a dosage based on the weight and age of the subject. In one embodiment, the step of administering a pharmaceutical composition further comprises the step of intravenously administering the composition. In one embodiment, when the subject is younger than 60 years of age, the dosage is 6 mg propofol per 10 kg, 2 mg ketamine per 10 kg, and 10-500 μg analgesic per 10 kg; when the subject is between 60 and 70 years of age, the dosage is 4.8 mg propofol per 10 kg, 1.6 mg ketamine per 10 kg, and 8-400 μg analgesic per 10 kg; and when the subject is over 70 years of age, the dosage is 3.6 mg propofol per 10 kg, 1.2 mg ketamine per 10 kg, and 6-300 μg analgesic per 10 kg. In one embodiment, when the subject is obese, the dosage is adjusted using the formula: Adjusted Weight for Dosing (AWFD)=Ideal Body Weight (IBW)+30% (actual weight−IBW). In one embodiment, the step of administering a pharmaceutical composition further comprises the step of mixing a solution of propofol with a solution of ketamine and a solution of analgesic to form a mixture. In one embodiment, the analgesic selected from the group consisting of alfentanil, fentanyl, remifentanil, hydromorphone, sufentanil, morphine, and dexmedetomidine. In one embodiment, the administering of a pharmaceutical composition further comprises the step of administering a solution of propofol when a maximum dosage of ketamine or analgesic is reached.

In one embodiment, when the subject is younger than 30 years of age, the dosage is 7.2 mg propofol per 10 kg, 2.4 mg ketamine per 10 kg, and 12-600 μg analgesic per 10 kg; and when the subject is over 85 years of age, the dosage is 3 mg propofol per 10 kg, 1 mg ketamine per 10 kg, and 5-250 μg analgesic per 10 kg. In one embodiment, when the subject is obese, the dosage is adjusted using the formula:

Adjusted Weight for Dosing (AWFD)=Ideal Body Weight (IBW)+30% (actual weight−IBW).

In one embodiment, the present invention further relates in part to a pharmaceutical composition comprising a solution of propofol, ketamine, and analgesic wherein the concentration of propofol 6 mg/mL, the concentration of ketamine is 2 mg/mL, and the concentration of analgesic is 10 μg/mL-500 μg/mL.

### DETAILED DESCRIPTION

It is to be understood that the descriptions of the present invention have been simplified to illustrate elements that are relevant for a clear understanding of the present invention, while eliminating, for the purpose of clarity, many other elements used in pharmaceutical compositions and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

As used herein, each of the following terms has the meaning associated with it in this section. Unless defined otherwise, all technical and scientific terms used herein generally have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “potency” refers to the dose needed to produce half the maximal response (ED₅₀).

As used herein, the term “efficacy” refers to the maximal effect (Eurax) achieved within an assay.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

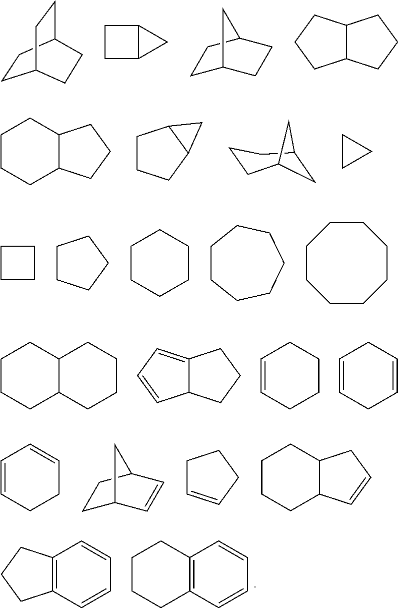

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

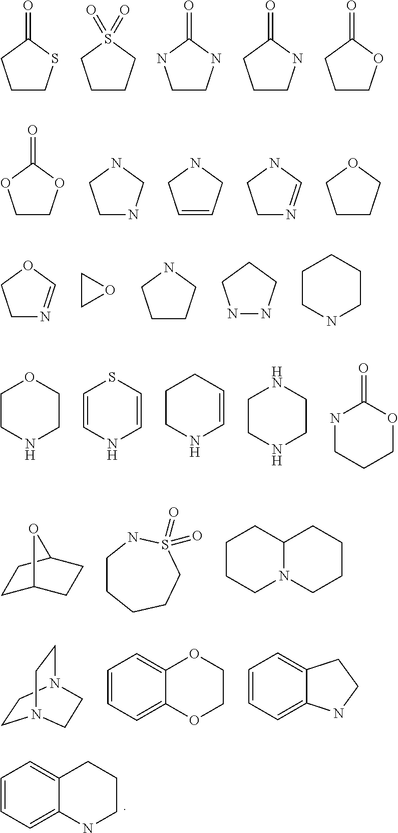

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. In one embodiment, aryl-(C₁-C₃)alkyl is aryl-CH₂— or aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

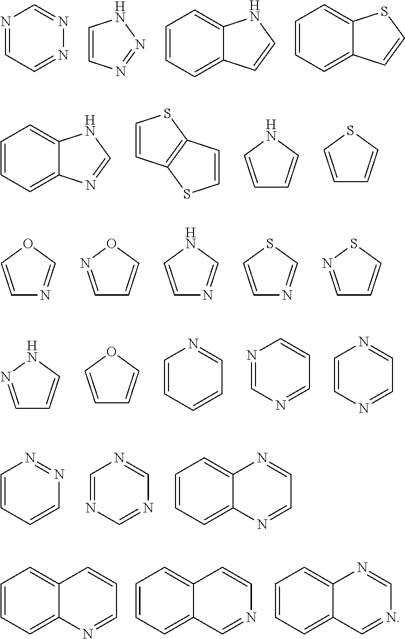

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, -N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Pharmaceutical Composition

The present invention relates in part to a pharmaceutical composition comprising propofol and ketamine. Such a pharmaceutical composition may consist of at least propofol, or a salt thereof, and ketamine, or a salt form thereof, in a form suitable for administration to a subject, or the pharmaceutical composition may comprise at least propofol, or a salt thereof, ketamine, or a salt form thereof, and one or more pharmaceutically acceptable carriers, one or more additional ingredients, or some combination of these. Propofol and ketamine may be present in the pharmaceutical composition in the form of a physiologically acceptable salt, such as in combination with a physiologically acceptable cation or anion, as is well known in the art.

In one embodiment, the pharmaceutical composition comprises 10 mg to 100 mg of propofol. In one embodiment, the pharmaceutical composition comprises 20 mg to 90 mg of propofol. In one embodiment, the pharmaceutical composition comprises 30 mg to 80 mg of propofol. In one embodiment, the pharmaceutical composition comprises 40 mg to 70 mg of propofol. In one embodiment, the pharmaceutical composition comprises 50 mg to 65 mg of propofol. In one embodiment, the pharmaceutical composition comprises 55 mg to 65 mg of propofol.

In one embodiment, the pharmaceutical composition comprises 1 mg to 100 mg of ketamine. In one embodiment, the pharmaceutical composition comprises 1 mg to 90 mg of ketamine. In one embodiment, the pharmaceutical composition comprises 1 mg to 80 mg of ketamine. In one embodiment, the pharmaceutical composition comprises 1 mg to 70 mg of ketamine. In one embodiment, the pharmaceutical composition comprises 1 mg to 60 mg of ketamine. In one embodiment, the pharmaceutical composition comprises 1 mg to 50 mg of ketamine. In one embodiment, the pharmaceutical composition comprises 1 mg to 40 mg of ketamine. In one embodiment, the pharmaceutical composition comprises 1 mg to 30 mg of ketamine. In one embodiment, the pharmaceutical composition comprises 10 mg to 30 mg of ketamine. In one embodiment, the pharmaceutical composition comprises 15 mg to 25 mg of ketamine.

In one embodiment, the pharmaceutical composition comprises at least one analgesic. The analgesic can be any analgesic known to a person of skill in the art. Exemplary analgesics include, but are not limited to, alfentanil, fentanyl, remifentanil, hydromorphone, sufentanil, codeine, hydrocodone, oxycodone, methadone, morphine, dihydromorphine, pethidine, buprenorphine, tramadol, tapentadol and dexmedetomidine. In one embodiment, the pharmaceutical composition comprises 0.02 mg to 1.5 mg of analgesic. In one embodiment, the pharmaceutical composition comprises 0.02 mg to 1.3 mg of analgesic. In one embodiment, the pharmaceutical composition comprises 0.02 mg to 1.1 mg of analgesic. In one embodiment, the pharmaceutical composition comprises 0.04 mg to 1.1 mg of analgesic. In one embodiment, the pharmaceutical composition comprises 0.06 mg to 1.1 mg of analgesic. In one embodiment, the pharmaceutical composition comprises 0.08 mg to 1.1 mg of analgesic. In one embodiment, the pharmaceutical composition comprises 0.09 mg to 1.1 mg of analgesic.

In one embodiment, the pharmaceutical composition is a liquid solution. The liquid may be any liquid known by a person of skill in the art. Exemplary liquids include, but are not limited to, water, deionized water, distilled water, sterilized water, saline solution, dextrose in water solution, and Ringer's lactate solution. In one embodiment, the milligram amounts disclosed elsewhere herein refer to the milligram amount of each of propofol, ketamine, and analgesic in a 10 mL liquid solution. In one embodiment, the amounts of each propofol, ketamine, and analgesic are doubled in a 20 mL liquid solution.

The concentration of propofol in the liquid solution can be any concentration known to a person of skill in the art. In one embodiment, the concentration of propofol in the liquid solution is between 100 μg/mL and 10 mg/mL. In one embodiment, the concentration of propofol in the liquid solution is between 150 μg/mL and 10 mg/mL. In one embodiment, the concentration of propofol in the liquid solution is between 200 μg/mL and 10 mg/mL. In one embodiment, the concentration of propofol in the liquid solution is between 250 μg/mL and 10 mg/mL. In one embodiment, the concentration of propofol in the liquid solution is between 300 μg/mL and 10 mg/mL. In one embodiment, the concentration of propofol in the liquid solution is between 350 μg/mL and 10 mg/mL. In one embodiment, the concentration of propofol in the liquid solution is between 400 μg/mL and 40 mg/mL. In one embodiment, the concentration of propofol in the liquid solution is between 450 μg/mL and 10 mg/mL. In one embodiment, the concentration of propofol in the liquid solution is between 500 μg/mL and 10 mg/mL. In one embodiment, the concentration of propofol in the liquid solution is between 550 μg/mL and 10 mg/mL. In one embodiment, the concentration of propofol in the liquid solution is between 600 μg/mL and 10 mg/mL. In one embodiment, the concentration of propofol in the liquid solution is between 650 μg/mL and 10 mg/mL. In one embodiment, the concentration of propofol in the liquid solution is between 700 μg/mL and 10 mg/mL. In one embodiment, the concentration of propofol in the liquid solution is between 750 μg/mL and 10 mg/mL. In one embodiment, the concentration of propofol in the liquid solution is between 800 μg/mL and 10 mg/mL. In one embodiment, the concentration of propofol in the liquid solution is between 850 μg/mL and 10 mg/mL. In one embodiment, the concentration of propofol in the liquid solution is between 900 μg/mL and 10 mg/mL. In one embodiment, the concentration of propofol in the liquid solution is between 950 μg/mL and 10 mg/mL. In one embodiment, the concentration of propofol in the liquid solution is between 1 mg/mL and 10 mg/mL. In one embodiment, the concentration of propofol in the liquid solution is between 1 mg/mL and 10 mg/mL. In one embodiment, the concentration of propofol in the liquid solution is between 5 mg/mL and 10 mg/mL. In one embodiment, the liquid solution comprises 6 mL of propofol at a concentration of 10 mg/mL.

In one embodiment, the concentration of ketamine in the liquid solution is between 100 μg/mL and 50 mg/mL. In one embodiment, the concentration of ketamine in the liquid solution is between 200 μg/mL and 50 mg/mL. In one embodiment, the concentration of ketamine in the liquid solution is between 300 μg/mL and 50 mg/mL. In one embodiment, the concentration of ketamine in the liquid solution is between 400 μg/mL and 50 mg/mL. In one embodiment, the concentration of ketamine in the liquid solution is between 500 μg/mL and 50 mg/mL. In one embodiment, the concentration of ketamine in the liquid solution is between 600 μg/mL and 50 mg/mL. In one embodiment, the concentration of ketamine in the liquid solution is between 700 μg/mL and 50 mg/mL. In one embodiment, the concentration of ketamine in the liquid solution is between 800 μg/mL and 50 mg/mL. In one embodiment, the concentration of ketamine in the liquid solution is between 900 μg/mL and 50 mg/mL. In one embodiment, the concentration of ketamine in the liquid solution is between 1 mg/mL and 50 mg/mL. In one embodiment, the concentration of ketamine in the liquid solution is between 1 mg/mL and 40 mg/mL. In one embodiment, the concentration of ketamine in the liquid solution is between 1 mg/mL and 30 mg/mL. In one embodiment, the concentration of ketamine in the liquid solution is between 1 mg/mL and 20 mg/mL. In one embodiment, the concentration of ketamine in the liquid solution is between 1 mg/mL and 10 mg/mL. In one embodiment, the concentration of ketamine in the liquid solution is between 1 mg/mL and 5 mg/mL. In one embodiment, the liquid solution comprises 2 mL of ketamine at a concentration of 10 mg/mL.

In one embodiment, the concentration of analgesic in the liquid solution is between 1 μg/mL and 100 mg/mL. In one embodiment, the concentration of analgesic in the liquid solution is between 1 μg/mL and 90 mg/mL. In one embodiment, the concentration of analgesic in the liquid solution is between 1 μg/mL and 80 mg/mL. In one embodiment, the concentration of analgesic in the liquid solution is between 1 μg/mL and 70 mg/mL. In one embodiment, the concentration of analgesic in the liquid solution is between 1 μg/mL and 60 mg/mL. In one embodiment, the concentration of analgesic in the liquid solution is between 1 μg/mL and 50 mg/mL. In one embodiment, the concentration of analgesic in the liquid solution is between 1 μg/mL and 40 mg/mL. In one embodiment, the concentration of analgesic in the liquid solution is between 1 μg/mL and 30 mg/mL. In one embodiment, the concentration of analgesic in the liquid solution is between 1 μg/mL and 20 mg/mL. In one embodiment, the concentration of analgesic in the liquid solution is between 1 μg/mL and 10 mg/mL. In one embodiment, the concentration of analgesic in the liquid solution is between 1 μg/mL and 5 mg/mL. In one embodiment, the concentration of analgesic in the liquid solution is between 1 μg/mL and 1 mg/mL. In one embodiment, the concentration of analgesic in the liquid solution is between 5 μg/mL and 0.5 mg/mL. In one embodiment, the concentration of analgesic in the liquid solution is between 5 μg/mL and 0.2 mg/mL. In one embodiment, the liquid solution comprises 2 mL of analgesic at a concentration of 0.05 mg/mL. In one embodiment, the liquid solution comprises 2 mL of analgesic at a concentration of 0.5 mg/mL.

In one embodiment, the pharmaceutical composition comprises 6 mL of 10 mg/mL propofol, 2 mL of 10 mg/mL ketamine, and 2 mL of 0.05 mg/mL analgesic. In one embodiment, the pharmaceutical composition comprises 6 mL of 10 mg/mL propofol, 2 mL of 10 mg/mL ketamine, and 2 mL of 0.5 mg/mL analgesic. In one embodiment, the pharmaceutical composition comprises 6 mL of 10 mg/mL propofol, 2 mL of 10 mg/mL ketamine, and 2 mL of 2.5 mg/mL analgesic. In one embodiment, the volume ratio of propofol to ketamine is 6:2 (in mL). In one embodiment, the volume ratio of propofol to analgesic is 6:2 (in mL). In one embodiment, the volume ratio of ketamine to analgesic is 2:2 (in mL). In one embodiment, the pharmaceutical composition comprises a volume ratio of 6 propofol: 2 ketamine: 2 analgesic (in mL).

In one embodiment, the weight ratio of propofol to ketamine in the pharmaceutical composition is between 1:1 and 100:1. In one embodiment, the weight ratio of propofol to ketamine in the pharmaceutical composition is between 1:1 and 90:1. In one embodiment, the weight ratio of propofol to ketamine in the pharmaceutical composition is between 1:1 and 80:1. In one embodiment, the weight ratio of propofol to ketamine in the pharmaceutical composition is between 1:1 and 70:1. In one embodiment, the weight ratio of propofol to ketamine in the pharmaceutical composition is between 1:1 and 60:1. In one embodiment, the weight ratio of propofol to ketamine in the pharmaceutical composition is between 1:1 and 50:1. In one embodiment, the weight ratio of propofol to ketamine in the pharmaceutical composition is between 1:1 and 40:1. In one embodiment, the weight ratio of propofol to ketamine in the pharmaceutical composition is between 1:1 and 30:1. In one embodiment, the weight ratio of propofol to ketamine in the pharmaceutical composition is between 1:1 and 20:1. In one embodiment, the weight ratio of propofol to ketamine in the pharmaceutical composition is between 1:1 and 10:1.

In one embodiment, the weight ratio of propofol to analgesic in the pharmaceutical composition is between 10:1 and 200:1. In one embodiment, the weight ratio of propofol to analgesic in the pharmaceutical composition is between 10:1 and 90:1. In one embodiment, the weight ratio of propofol to analgesic in the pharmaceutical composition is between 10:1 and 80:1. In one embodiment, the weight ratio of propofol to analgesic in the pharmaceutical composition is between 10:1 and 70:1. In one embodiment, the weight ratio of propofol to analgesic in the pharmaceutical composition is between 20:1 and 70:1. In one embodiment, the weight ratio of propofol to analgesic in the pharmaceutical composition is between 30:1 and 70:1. In one embodiment, the weight ratio of propofol to analgesic in the pharmaceutical composition is between 40:1 and 70:1. In one embodiment, the weight ratio of propofol to analgesic in the pharmaceutical composition is between 50:1 and 70:1. In one embodiment, the weight ratio of propofol to analgesic in the pharmaceutical composition is between 55:1 and 65:1.

In one embodiment, the weight ratio of propofol to analgesic in the pharmaceutical composition is between 100:1 and 1500:1. In one embodiment, the weight ratio of propofol to analgesic in the pharmaceutical composition is between 100:1 and 1400:1. In one embodiment, the weight ratio of propofol to analgesic in the pharmaceutical composition is between 100:1 and 1300:1. In one embodiment, the weight ratio of propofol to analgesic in the pharmaceutical composition is between 100:1 and 1200:1. In one embodiment, the weight ratio of propofol to analgesic in the pharmaceutical composition is between 100:1 and 1100:1. In one embodiment, the weight ratio of propofol to analgesic in the pharmaceutical composition is between 100:1 and 1000:1. In one embodiment, the weight ratio of propofol to analgesic in the pharmaceutical composition is between 100:1 and 900:1. In one embodiment, the weight ratio of propofol to analgesic in the pharmaceutical composition is between 100:1 and 800:1. In one embodiment, the weight ratio of propofol to analgesic in the pharmaceutical composition is between 100:1 and 700:1. In one embodiment, the weight ratio of propofol to analgesic in the pharmaceutical composition is between 200:1 and 700:1. In one embodiment, the weight ratio of propofol to analgesic in the pharmaceutical composition is between 300:1 and 700:1. In one embodiment, the weight ratio of propofol to analgesic in the pharmaceutical composition is between 400:1 and 700:1. In one embodiment, the weight ratio of propofol to analgesic in the pharmaceutical composition is between 500:1 and 700:1. In one embodiment, the weight ratio of propofol to analgesic in the pharmaceutical composition is between 550:1 and 650:1.

In one embodiment, the weight ratio of ketamine to analgesic in the pharmaceutical composition is between 1:1 and 150:1. In one embodiment, the weight ratio of ketamine to analgesic in the pharmaceutical composition is between 1:1 and 140:1. In one embodiment, the weight ratio of ketamine to analgesic in the pharmaceutical composition is between 1:1 and 130:1. In one embodiment, the weight ratio of ketamine to analgesic in the pharmaceutical composition is between 1:1 and 120:1. In one embodiment, the weight ratio of ketamine to analgesic in the pharmaceutical composition is between 1:1 and 110:1. In one embodiment, the weight ratio of ketamine to analgesic in the pharmaceutical composition is between 1:1 and 100:1. In one embodiment, the weight ratio of ketamine to analgesic in the pharmaceutical composition is between 1:1 and 90:1. In one embodiment, the weight ratio of ketamine to analgesic in the pharmaceutical composition is between 1:1 and 80:1. In one embodiment, the weight ratio of ketamine to analgesic in the pharmaceutical composition is between 1:1 and 70:1. In one embodiment, the weight ratio of ketamine to analgesic in the pharmaceutical composition is between 1:1 and 60:1. In one embodiment, the weight ratio of ketamine to analgesic in the pharmaceutical composition is between 1:1 and 50:1. In one embodiment, the weight ratio of ketamine to analgesic in the pharmaceutical composition is between 1:1 and 40:1. In one embodiment, the weight ratio of ketamine to analgesic in the pharmaceutical composition is between 1:1 and 30:1. In one embodiment, the weight ratio of ketamine to analgesic in the pharmaceutical composition is between 10:1 and 30:1. In one embodiment, the weight ratio of ketamine to analgesic in the pharmaceutical composition is between 15:1 and 25:1.

In one embodiment, the weight ratio of ketamine to analgesic in the pharmaceutical composition is between 1:1 and 1000:1. In one embodiment, the weight ratio of ketamine to analgesic in the pharmaceutical composition is between 1:1 and 900:1. In one embodiment, the weight ratio of ketamine to analgesic in the pharmaceutical composition is between 1:1 and 800:1. In one embodiment, the weight ratio of ketamine to analgesic in the pharmaceutical composition is between 1:1 and 700:1. In one embodiment, the weight ratio of ketamine to analgesic in the pharmaceutical composition is between 1:1 and 600:1. In one embodiment, the weight ratio of ketamine to analgesic in the pharmaceutical composition is between 1:1 and 500:1. In one embodiment, the weight ratio of ketamine to analgesic in the pharmaceutical composition is between 1:1 and 400:1. In one embodiment, the weight ratio of ketamine to analgesic in the pharmaceutical composition is between 1:1 and 300:1. In one embodiment, the weight ratio of ketamine to analgesic in the pharmaceutical composition is between 100:1 and 300:1. In one embodiment, the weight ratio of ketamine to analgesic in the pharmaceutical composition is between 150:1 and 250:1. In one embodiment, the weight ratio of ketamine to analgesic in the pharmaceutical composition is between 175:1 and 225:1.

In one embodiment, the volume ratio of propofol to ketamine in the pharmaceutical composition is between 1:1 and 50:1. In one embodiment, the volume ratio of propofol to ketamine in the pharmaceutical composition is between 1:1 and 45:1. In one embodiment, the volume ratio of propofol to ketamine in the pharmaceutical composition is between 1:1 and 40:1. In one embodiment, the volume ratio of propofol to ketamine in the pharmaceutical composition is between 1:1 and 35:1. In one embodiment, the volume ratio of propofol to ketamine in the pharmaceutical composition is between 1:1 and 30:1. In one embodiment, the volume ratio of propofol to ketamine in the pharmaceutical composition is between 1:1 and 25:1. In one embodiment, the volume ratio of propofol to ketamine in the pharmaceutical composition is between 1:1 and 20:1. In one embodiment, the volume ratio of propofol to ketamine in the pharmaceutical composition is between 1:1 and 15:1. In one embodiment, the volume ratio of propofol to ketamine in the pharmaceutical composition is between 1:1 and 5:1.

In one embodiment, the volume ratio of propofol to analgesic in the pharmaceutical composition is between 1:1 and 50:1. In one embodiment, the volume ratio of propofol to analgesic in the pharmaceutical composition is between 1:1 and 45:1. In one embodiment, the volume ratio of propofol to analgesic in the pharmaceutical composition is between 1:1 and 40:1. In one embodiment, the volume ratio of propofol to analgesic in the pharmaceutical composition is between 1:1 and 35:1. In one embodiment, the volume ratio of propofol to analgesic in the pharmaceutical composition is between 1:1 and 30:1. In one embodiment, the volume ratio of propofol to analgesic in the pharmaceutical composition is between 1:1 and 25:1. In one embodiment, the volume ratio of propofol to analgesic in the pharmaceutical composition is between 1:1 and 20:1. In one embodiment, the volume ratio of propofol to analgesic in the pharmaceutical composition is between 1:1 and 15:1. In one embodiment, the volume ratio of propofol to analgesic in the pharmaceutical composition is between 1:1 and 5:1.

In one embodiment, the volume ratio of ketamine to analgesic in the pharmaceutical composition is between 1:1 and 50:1. In one embodiment, the volume ratio of ketamine to analgesic in the pharmaceutical composition is between 1:1 and 45:1. In one embodiment, the volume ratio of ketamine to analgesic in the pharmaceutical composition is between 1:1 and 40:1. In one embodiment, the volume ratio of ketamine to analgesic in the pharmaceutical composition is between 1:1 and 35:1. In one embodiment, the volume ratio of ketamine to analgesic in the pharmaceutical composition is between 1:1 and 30:1. In one embodiment, the volume ratio of ketamine to analgesic in the pharmaceutical composition is between 1:1 and 25:1. In one embodiment, the volume ratio of ketamine to analgesic in the pharmaceutical composition is between 1:1 and 20:1. In one embodiment, the volume ratio of ketamine to analgesic in the pharmaceutical composition is between 1:1 and 15:1. In one embodiment, the volume ratio of ketamine to analgesic in the pharmaceutical composition is between 1:1 and 5:1.

Method of Treating a Subject with the Pharmaceutical Composition

The present invention also relates to a method of providing sedation to a subject in need thereof, the method comprising the step of administering to the subject a pharmaceutical composition comprising a solution of propofol and ketamine. The present invention comprises a method of providing sedation to a subject in surgical need thereof, the method comprising the step of administering to the subject a pharmaceutical composition comprising a solution of propofol and ketamine. The pharmaceutical composition may comprise any active ingredients, solvents, excipients, carriers, or “additional” ingredients described elsewhere herein.

In one embodiment, the pharmaceutical composition is administered in a dosage based on the weight and age of the subject. In one embodiment, the liquid pharmaceutical composition is delivered to a patient younger than 30 years of age at a dosage of 1.2 mL per 10 kg of body weight. In one embodiment, the 1.2 mL dosage per 10 kg of body weight administered to a patient younger than 30 years of age comprises 7.2 mg propofol per 10 kg, 2.4 mg ketamine per 10 kg, and 12-600 μg analgesic per 10 kg of body weight. In one embodiment, the liquid pharmaceutical composition is delivered at this dosage via a bolus dose.

In one embodiment, the liquid pharmaceutical composition is delivered to a patient between 30 and 60 years of age at a dosage of 1 mL per 10 kg of body weight. In one embodiment, 1 mL dosage per 10 kg of body weight administered to a patient between 30 and 60 years of age comprises 6 mg propofol per 10 kg, 2 mg ketamine per 10 kg, and 10-500 μg analgesic per 10 kg of body weight. In one embodiment, the liquid pharmaceutical composition is delivered at this dosage via a bolus dose.

In one embodiment, the liquid pharmaceutical composition is delivered to a patient between 60 and 70 years of age at a dosage of 0.8 mL per 10 kg of body weight. In one embodiment, the 0.8 mL dosage per 10 kg of body weight administered to a patient between 60 and 70 years of age comprises 4.8 mg propofol per 10 kg, 1.6 mg ketamine per 10 kg, and 8-400 μg analgesic per 10 kg of body weight. In one embodiment, the liquid pharmaceutical composition is delivered at this dosage via a bolus dose.

In one embodiment, the liquid pharmaceutical composition is delivered to a patient between 70 and 85 years of age at a dosage of 0.6 mL per 10 kg of body weight.

In one embodiment, the 0.6 mL dosage per 10 kg of body weight administered to a patient between 70 and 85 years of age comprises 3.6 mg propofol per 10 kg, 1.2 mg ketamine per 10 kg, and 6-300 μg analgesic per 10 kg of body weight. In one embodiment, the liquid pharmaceutical composition is delivered at this dosage via a bolus dose.

In one embodiment, the liquid pharmaceutical composition is delivered to a patient older than 85 years of age at a dosage of 0.5 mL per 10 kg of body weight. In one embodiment, the 0.5 mL dosage per 10 kg of body weight administered to a patient older than 85 years of age comprises 3 mg propofol per 10 kg, 1 mg ketamine per 10 kg, and 5-250 μg analgesic per 10 kg of body weight. In one embodiment, the liquid pharmaceutical composition is delivered at this dosage via a bolus dose.

For example, a 50-year-old female, height 5′2″, weight 50 kg, Ideal Body Weight (IBW) 50.2 kg. The bolus dose will be 5 mL.

In one embodiment, the dosages described elsewhere herein are adjusted for administration to an obese patient, having a Body Mass Index (BMI) >30, using the following formula: Adjusted Weight for Dosing (AWFD)=Ideal Body Weight (IBW)+30% (actual weight−IBW). In one embodiment, the pharmaceutical composition is administered to the patient immediately before nerve blocks. In one embodiment, a bolus dose of the pharmaceutical composition is adjusted using the above formula before administration to an obese patient. For example, a 50-year-old male, height 5′11″, weight 125 kg, IBW 75.3 kg. The difference between his actual weight and IBW is 50 kg. 30% of the 50 kg is 15 kg. Therefore, the AWFD for this man is 75.3+15=90 kg. The bolus dose is 9 mL.

In one embodiment, the pharmaceutical composition is delivered all at once. In one embodiment, the pharmaceutical composition is delivered as a bolus dose. In one embodiment, the bolus dose is delivered via infusion pump. In one embodiment, the bolus dose is delivered via syringe. In one embodiment, the syringe is pushed by hand. In one embodiment, the pharmaceutical composition is delivered as a bolus dose immediately before nerve blocks. In one embodiment, the pharmaceutical composition is delivered via a continuous infusion. In one embodiment, the pharmaceutical composition is delivered via a continuous infusion (after bolus dose and nerve blocks, then during surgery). In one embodiment, the composition is delivered via both a bolus dose and a continuous infusion. In one embodiment, the composition is delivered intravenously.

In one embodiment, the pharmaceutical composition is administered to a patient continuously. In one embodiment, the continuous dose is administered to a patient after the bolus dose. In one embodiment, the pharmaceutical composition is administered continuously after nerve blocks. In one embodiment, the pharmaceutical composition is administered continuously after both the bolus dose and nerve blocks. In one embodiment, the pharmaceutical composition is administered to a patient during surgery. In one embodiment, the pharmaceutical composition is administered to a patient intravenously.

In one embodiment, the infusion rate of the pharmaceutical composition is between 1 mL/hr and 50 mL/hr. In one embodiment, the infusion rate of the pharmaceutical composition is between 1 mL/hr and 45 mL/hr. In one embodiment, the infusion rate of the pharmaceutical composition is between 1 mL/hr and 40 mL/hr. In one embodiment, the infusion rate of the pharmaceutical composition is between 1 mL/hr and 35 mL/hr. In one embodiment, the infusion rate of the pharmaceutical composition is between 1 mL/hr and 30 mL/hr. In one embodiment, the infusion rate of the pharmaceutical composition is between 5 mL/hr and 25 mL/hr.

In one embodiment, the infusion rate of the pharmaceutical composition will be determined by the patient's age, weight, or anxiety level. In one embodiment, the infusion rate of the pharmaceutical composition will be determined by duration or extensiveness of the surgery. In one embodiment, the infusion rate of the pharmaceutical composition will be determined by more than one of the above factors.

In one embodiment, a continuous dose of between 5 mg and 200 mg of propofol is administered to a patient per hour. In one embodiment, a continuous dose of between 5 mg and 190 mg of propofol is administered to a patient per hour. In one embodiment, a continuous dose of between 5 mg and 180 mg of propofol is administered to a patient per hour. In one embodiment, a continuous dose of between 5 mg and 170 mg of propofol is administered to a patient per hour. In one embodiment, a continuous dose of between 5 mg and 160 mg of propofol is administered to a patient per hour. In one embodiment, a continuous dose of between 5 mg and 150 mg of propofol is administered to a patient per hour. In one embodiment, a continuous dose of between 15 mg and 150 mg of propofol is administered to a patient per hour. In one embodiment, a continuous dose of between 25 mg and 150 mg of propofol is administered to a patient per hour. In one embodiment, a continuous dose of between 30 mg and 150 mg of propofol is administered to a patient per hour.

In one embodiment, a continuous dose of between 1 mg and 100 mg of ketamine is administered to a patient per hour. In one embodiment, a continuous dose of between 1 mg and 90 mg of ketamine is administered to a patient per hour. In one embodiment, a continuous dose of between 1 mg and 80 mg of ketamine is administered to a patient per hour. In one embodiment, a continuous dose of between 1 mg and 70 mg of ketamine is administered to a patient per hour. In one embodiment, a continuous dose of between 1 mg and 60 mg of ketamine is administered to a patient per hour. In one embodiment, a continuous dose of between 5 mg and 55 mg of ketamine is administered to a patient per hour.

In one embodiment, a continuous dose of between 0.1 mg and 3 mg of analgesic is administered to a patient per hour. In one embodiment, a continuous dose of between 0.1 mg and 2.9 mg of analgesic is administered to a patient per hour. In one embodiment, a continuous dose of between 0.1 mg and 2.8 mg of analgesic is administered to a patient per hour. In one embodiment, a continuous dose of between 0.1 mg and 2.7 mg of analgesic is administered to a patient per hour. In one embodiment, a continuous dose of between 0.1 mg and 2.6 mg of analgesic is administered to a patient per hour. In one embodiment, a continuous dose of between 0.1 mg and 2.5 mg of analgesic is administered to a patient per hour. In one embodiment, a continuous dose of between 0.2 mg and 2.5 mg of analgesic is administered to a patient per hour. In one embodiment, a continuous dose of between 0.3 mg and 2.5 mg of analgesic is administered to a patient per hour. In one embodiment, a continuous dose of between 0.4 mg and 2.5 mg of analgesic is administered to a patient per hour. In one embodiment, a continuous dose of between 0.5 mg and 2.5 mg of analgesic is administered to a patient per hour. In one embodiment, a continuous dose of between 0.55 mg and 2.45 mg of analgesic is administered to a patient per hour.

In one embodiment, a continuous dose of between 1 μg and 100 μg of analgesic is administered to a patient per hour. In one embodiment, a continuous dose of between 10 μg and 300 μg of analgesic is administered to a patient per hour. In one embodiment, a continuous dose of between 10 μg and 290 μg of analgesic is administered to a patient per hour. In one embodiment, a continuous dose of between 10 μg and 280 μg of analgesic is administered to a patient per hour. In one embodiment, a continuous dose of between 10 μg and 270 μg of analgesic is administered to a patient per hour. In one embodiment, a continuous dose of between 10 μg and 260 μg of analgesic is administered to a patient per hour. In one embodiment, a continuous dose of between 10 μg and 250 μg of analgesic is administered to a patient per hour. In one embodiment, a continuous dose of between 20 μg and 250 μg of analgesic is administered to a patient per hour. In one embodiment, a continuous dose of between 30 μg and 250 μg of analgesic is administered to a patient per hour. In one embodiment, a continuous dose of between 40 μg and 250 μg of analgesic is administered to a patient per hour. In one embodiment, a continuous dose of between 50 μg and 250 μg of analgesic is administered to a patient per hour. In one embodiment, a continuous dose of between 55 μg and 245 μg of analgesic is administered to a patient per hour.

In one embodiment, the continuous dose is administered until surgery is complete. In one embodiment, the continuous dose is administered until a maximum dose of ketamine or analgesic is reached. In one embodiment, the maximum dosage of ketamine is between 50 mg and 150 mg. In one embodiment, the maximum dosage of ketamine is between 50 mg and 140 mg. In one embodiment, the maximum dosage of ketamine is between 50 mg and 130 mg. In one embodiment, the maximum dosage of ketamine is between 50 mg and 120 mg. In one embodiment, the maximum dosage of ketamine is between 50 mg and 110 mg. In one embodiment, the maximum dosage of ketamine is between 60 mg and 110 mg. In one embodiment, the maximum dosage of ketamine is between 70 mg and 110 mg. In one embodiment, the maximum dosage of ketamine is between 80 mg and 110 mg. In one embodiment, the maximum dosage of ketamine is between 90 mg and 110 mg. In one embodiment, the maximum dosage of ketamine is between 95 mg and 110 mg.

In one embodiment, the continuous dose is administered until a maximum dosage of fentanyl is reached. In one embodiment, the maximum dosage of fentanyl is between 450 μg and 550 μg. In one embodiment, the maximum dosage of fentanyl is between 450 μg and 540 μg. In one embodiment, the maximum dosage of fentanyl is between 450 μg and 530 μg. In one embodiment, the maximum dosage of fentanyl is between 450 μg and 520 μg. In one embodiment, the maximum dosage of fentanyl is between 450 μg and 510 μg. In one embodiment, the maximum dosage of fentanyl is between 460 μg and 510 μg. In one embodiment, the maximum dosage of fentanyl is between 470 μg and 510 μg. In one embodiment, the maximum dosage of fentanyl is between 480 μg and 510 μg. In one embodiment, the maximum dosage of fentanyl is between 490 μg and 510 μg. In one embodiment, the maximum dosage of fentanyl is between 495 μg and 510 μg.

In one embodiment, the continuous dose is administered until a maximum dosage of an analgesic other than fentanyl is reached. The analgesic may be any analgesic described elsewhere herein. In one embodiment, the analgesic is alfentanil and the maximum dosage is between 1.95 mg and 2.1 mg. In one embodiment, the analgesic is remifentanil the maximum dosage of is between 1.95 mg and 2.1 mg. In one embodiment, the analgesic is hydromorphone and the maximum dosage is between 1.95 mg and 2.1 mg. In one embodiment, the analgesic is sufentanil and the maximum dosage is between 495 μg and 510 μg. In one embodiment, the analgesic is morphine and the maximum dosage is between 9.9 mg and 10.1 mg. In one embodiment, the analgesic is morphine and the maximum dosage is between 18 mg and 22 mg. In one embodiment, the analgesic is dexmedetomidine and the maximum dosage is between 0.95 mg and 1.1 mg.

In one embodiment, when the maximum dosage of ketamine or analgesic is reached, a solution propofol without ketamine or analgesic is administered to the patient. In one embodiment, the solution of propofol is administered continuously. In one embodiment, the propofol is administered intravenously. In one embodiment, the solution of propofol is administered from when the maximum dosage of ketamine or analgesic is reached until the end of surgery. In one embodiment, propofol is administered at a dosage of between 1 and 200 μg/kg/min. In one embodiment, propofol is administered at a dosage of between 1 and 190 μg/kg/min. In one embodiment, propofol is administered at a dosage of between 1 and 180 μg/kg/min. In one embodiment, propofol is administered at a dosage of between 1 and 170 μg/kg/min. In one embodiment, propofol is administered at a dosage of between 1 and 160 μg/kg/min. In one embodiment, propofol is administered at a dosage of between 1 and 150 μg/kg/min. In one embodiment, propofol is administered at a dosage of between 1 and 140 μg/kg/min. In one embodiment, propofol is administered at a dosage of between 1 and 130 μg/kg/min. In one embodiment, propofol is administered at a dosage of between 1 and 120 μg/kg/min. In one embodiment, propofol is administered at a dosage of between 1 and 110 μg/kg/min. In one embodiment, propofol is administered at a dosage of between 10 and 110 μg/kg/min. In one embodiment, propofol is administered at a dosage of between 20 and 110 μg/kg/min. In one embodiment, propofol is administered at a dosage of between 20 and 105 μg/kg/min.

### Pharmaceutical Compositions and Formulations

The relative amounts of the propofol and ketamine, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) propofol and ketamine. The composition may comprise between 0.1% and 100% (w/w) of one or more additional active ingredients.

In one embodiment, the compositions of the invention comprise one or more pharmaceutically acceptable excipients or carriers. In one embodiment, the pharmaceutical compositions of the invention comprise a therapeutically effective amount of propofol, ketamine, and a pharmaceutically acceptable carrier. In one embodiment, the pharmaceutical compositions of the invention comprise a therapeutically effective amount of propofol, ketamine, an analgesic, and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition may comprise any form known to a person of skill in the art. In one embodiment, the pharmaceutical composition comprises a liquid suspension of one of more of the active ingredients in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In one embodiment, the pharmaceutical composition comprises a liquid solution of the one or more active ingredients in aqueous or oily solvents, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

In one embodiment, the pharmaceutical composition comprises a powdered or granular formulation of the one or more active ingredients. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

In one embodiment, the pharmaceutical composition of the invention may be in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

In an embodiment, the pharmaceutical compositions may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Pharmaceutical compositions that are useful in the methods of the invention may be suitably developed for oral, rectal, vaginal, parenteral, topical, pulmonary, intranasal, buccal, ophthalmic, intravenous, or another route of administration. A composition useful within the methods of the invention may be directly administered to the skin, or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3,050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, parenteral, sublingual, transdermal, transmucosal (e.g., sublingual, lingual, (trans)buccal, and (intra)nasal,), intravesical, intraduodenal, intragastrical, rectal, intra-peritoneal, subcutaneous, intramuscular, intradermal, intra-arterial, intravenous, or combinations of one or more types of administration.

Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Materials and Methods

Concentration of stock solutions: Propofol and ketamine stock solutions are each 10 mg/mL. The concentrations of the analgesic stock solutions vary depending on the analgesic selected. For example, alfentanil is 0.5 mg/mL, fentanyl is 0.05 mg/mL, and hydromorphone is 1 or 2 mg/mL. Remifentanil stock is formulated as powder (1 or 2 mg) from the manufacturer and is usually dissolved in sterile water to final concentration of 0.05 mg/mL. The stock concentration of morphine is 10 mg/mL, and of dexmedetomidine is 100 μg/mL, respectively.
Preparation of the pharmaceutical composition: In a 10 mL syringe, 6 mL of propofol, 2 mL of ketamine, and 2 mL of fentanyl are added, forming a 6-2-2 mixture known as KF6-2-2. The solution is mixed well. Other pharmaceutical compositions are obtained by varying the analgesic, the KA6-2-2 mixture comprises alfentanil, the KR6-2-2 mixture comprises remifentanil, the KH6-2-2 mixture comprises hydromorphone, the KS6-2-2 mixture comprises sufentanil, the KM6-2-2 mixture comprises morphine, and the KD6-2-2 mixture comprises dexmedetomidine.
Administration of the pharmaceutical composition: Before nerve blocks are performed, a bolus of the mixture is delivered by infusion pump or hand push approximately over 5 seconds. The dose of the mixture is based on patient's weight and age. Usually, patient will reach OAA/S (Observer's Assessment of Alertness/Sedation) score 3 level of sedation (slow verbal response to verbal stimulation, facial relaxation, and eyes close spontaneously) in less than one minute and is then ready for nerve blocks (retrobulbar block, ankle blocks, or injection of local anesthetics). The efficacy of painlessness is 90% or higher. Most of the patients are still be able to respond to verbal stimulation and breathe in response to demand without apnea or oxygen desaturation. Although some patients can still talk during the blocks, most of them will not recall the blocks. The incidence of recall of injection is <20%.

### Results and Discussion

The common practice of intravenous sedation for surgery is to administer several medications in different syringes to reduce anxiety (anxielytics), prevent pain associated with surgery (narcotics), provide continuous sedation (propofol), and prevent pain caused by propofol (lidocaine). It is very cumbersome to titrate all these medications simultaneously and the sedative effects on patients are often unpredictable. This type of technique is not only time consuming, but also unsafe since it is prone to oversedation or undersedation, leading to airway complications or injury if patients move during surgery, e.g., during retrobulbar blocks.

The present invention is a new pharmaceutical composition for intravenous sedation that is both efficacious and safe. The composition is made by mixing 6 mL of propofol, 2 mL of ketamine, and 2 mL of fentanyl to form a 6-2-2 mixture known as KF6-2-2. The composition is delivered to patients in a bolus dose using an infusion pump or hand pump, such that 1 mL per 10 kg (patient's weight) of the solution is given to a patient younger than 60 years of age. In patients that are 60 to 70 years of age, the above dose is decreased by 20%. In patients older than 70 years of age, the above dose is decreased by 40%. If a patient is older than 85 years of age, the bolus dose will be further decreased 50%. However, if a patient is younger than 30 years of age, the bolus dose can be increased 20% to 1.2 mL per 10 kg, including 7.2 mg propofol per 10 kg, 2.4 mg ketamine per 10 kg, and 12-600 μg analgesic per 10 kg.

For example, a 50-year-old female, height 5′2″, weight 50 kg, IBW 50.2 kg. The bolus dose will be 5 mL.

When the patient is obese, the dosage is adjusted using the formula:

Adjusted Weight for Dosing (AWFD)=Ideal Body Weight (IBW)+30% (actual weight−IBW).

For example, a 50-year-old male, height 5′11″, weight 125 kg, IBW 75.3 kg. The difference between his actual weight and IBW is 50 kg. 30% of the 50 kg is 15 kg. Therefore, the AWFD for this man is 75.3+15=90 kg. The bolus dose is 9 mL.

In general, the 10 mL mixture provides sedation for procedures less than 30 minutes. For longer surgeries, the volume can be increased 20 or 40 mL to cover up to 3-5 hours of sedation, depending on a patient's age and weight.

During surgery, the infusion rate of the mixture will be between 6-24 mL/hour determined by subject's age, weight, anxiety level, duration and the extensiveness of surgery. In general, the total dose of analgesics is limited as following: 2 mg for alfentanil; 500 μg for fentanyl; 2 mg for remifentanil; 2 mg for hydromorphone; and 500 μg for sufentanil; 20 mg for morphine; and 1 mg for dexmedetomidine. The total dose of ketamine is limited to 100 mg. When the maximum dose of ketamine and analgesics is reached, the intravenous sedation will be switched to propofol only at 25-100 μg/kg/min until the end of surgery.

The KF6-2-2 technique represents a novel way to conduct intravenous sedation in a quantitatively controllable and predictable fashion in terms of its efficacy, timing, and incidence of complications. In comparison to conventional sedation techniques, the time to reach target level of sedation for the KF6-2-2 technique is one minute or less compared to 10 to 20 minutes for the conventional method. 90% of patients do not experience pain with the KF (or other analgesics) 6-2-2 technique compared to 30-60% of patients having unpredictable pain control in conventional sedation and less than 2% of patients required intervention for airway complications with the KF6-2-2 technique versus 30-50% in conventional sedation. The bolus dose of the KF6-2-2 mixture can also prevent head or body movement (which is a risk factor for eye injury during eye blocks) in most of the patients during nerve blocks; however, head and body movement is common during nerve blocks with the conventional sedation. In general, a patient's blood pressure and heart rate remain in the normal range or within 20% from their baseline blood pressure. The KF6-2-2 technique requires only one syringe for the preparation and shows reproducible sedation and analgesic results in almost every patient.

The potential financial economic gain from the clinical use of the invention could be very significant because the technique is very safe with very low risk of complications. Additionally, this inventive technique is very efficient, resulting in short turn-over time between cases (save 10 to 15 minutes on average per case, with $60-80 per minute expense for operating room time), thus allowing for the scheduling of more cases within the regular operating room (OR) time. Lastly, this inventive technique is excellent in efficacy in providing great patient comfort and painlessness during nerve blocks and surgery, resulting in great patient satisfaction.

The invention can also be easily compounded and commercialized with standardization in dosing and delivering by infusion pump for almost every patient undergoing surgical procedures. However, this technique(s) should be used only by trained anesthesiologist or nurse anesthetist.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A pharmaceutical composition comprising a solution of propofol, ketamine, and an analgesic wherein the concentration of propofol is 1 mg/mL-10 mg/mL, the concentration of ketamine is 0.1 mg/mL-10 mg/mL, and the concentration of analgesic is 1 μg/mL-1 mg/mL.

2. The pharmaceutical composition of claim 1, wherein the analgesic is selected from the group consisting of alfentanil, fentanyl, remifentanil, hydromorphone, sufentanil, morphine, and dexmedetomidine.

3. The pharmaceutical composition of claim 1, wherein the analgesic is fentanyl.

4. The pharmaceutical composition of claim 1, wherein the concentration of propofol is 5 mg/mL-7 mg/mL, the concentration of ketamine is 1 mg/mL-3 mg/mL, and the concentration of analgesic is 5 μg/mL-0.15 mg/mL.

5. The pharmaceutical composition of claim 1, wherein the solution comprises a sterile medium selected from the group consisting of water, deionized water, distilled water, sterilized water, saline solution, dextrose in water solution, and Ringer's lactate solution.

6. A method of providing sedation to a subject in need thereof, the method comprising the step of
administering to the subject a pharmaceutical composition comprising a solution of propofol, ketamine, and an analgesic wherein the concentration of propofol is 1 mg/mL-10 mg/mL, the concentration of ketamine is 0.1 mg/mL-10 mg/mL, and the concentration of analgesic is 1 μg/mL-1 mg/mL; and wherein the composition is administered in a dosage based on the weight and age of the subject.

7. The method of claim 6, wherein the step of administering a pharmaceutical composition further comprises the step of intravenously administering the composition.

8. The method of claim 6, wherein when the pharmaceutical composition is administered as a bolus dose and the subject is younger than 60 years of age, the dosage is 6 mg propofol per 10 kg, 2 mg ketamine per 10 kg, and 10-500 μg analgesic per 10 kg; when the subject is between 60 and 70 years of age, the dosage is 4.8 mg propofol per 10 kg, 1.6 mg ketamine per 10 kg, and 8-400 μg analgesic per 10 kg; and when the subject is over 70 years of age, the dosage is 3.6 mg propofol per 10 kg, 1.2 mg ketamine per 10 kg, and 6-300 μg analgesic per 10 kg.

9. The method of claim 8, wherein when the subject is obese, the dosage is adjusted using the formula:
Adjusted Weight for Dosing (AWFD)=Ideal Body Weight (IBW)+30% (actual weight−IBW).

10. The method of claim 6, wherein the step of administering a pharmaceutical composition further comprises the step of mixing a solution of propofol with a solution of ketamine and a solution of analgesic to form a mixture.

11. The method of claim 6, wherein the analgesic selected from the group consisting of alfentanil, fentanyl, remifentanil, hydromorphone, sufentanil, morphine, and dexmedetomidine.

12. The method of claim 6, wherein the of administering a pharmaceutical composition further comprises the step of administering a solution of propofol when a maximum dosage of ketamine or analgesic is reached.

13. The method of claim 6, wherein when the pharmaceutical composition is administered as a bolus dose and the subject is younger than 30 years of age, the dosage is 7.2 mg propofol per 10 kg, 2.4 mg ketamine per 10 kg, and 12-600 μg analgesic per 10 kg; and when the subject is over 85 years of age, the dosage is 3 mg propofol per 10 kg, 1 mg ketamine per 10 kg, and 5-250 μg analgesic per 10 kg.

14. The method of claim 13, wherein when the subject is obese, the dosage is adjusted using the formula:
Adjusted Weight for Dosing (AWFD)=Ideal Body Weight (IBW)+30% (actual weight−IBW).

15. A pharmaceutical composition comprising a solution of propofol, ketamine, and analgesic wherein the concentration of propofol 6 mg/mL, the concentration of ketamine is 2 mg/mL, and the concentration of analgesic is 10 μg/mL-500 μg/mL.
